(19) United States
(12) Patent Application Publication
(10) Pub. No.: US 2002/0058909 A1
(43) Pub. Date: May 16, 2002

(54) Mixing apparatus

(76) Inventors: Richard J. Deslauriers, Woodbury, CT (US); Robert T. Potash, South Windsor, CT (US); Stephanie Bonin, West Hartford, CT (US)

Correspondence Address: Richard J. Deslauriers, M.D., 50 Altair Avenue, Plymouth, CT, 06782, US

(21) Appl. No.: 10/036,186

(22) Filed: Dec. 28, 2001

Related U.S. Application Data
(63) Continuation-in-part of application No. 09/877,171, filed on Jun. 11, 2001.
(60) Provisional application No. 60/241,434, filed on Oct. 19, 2000.

(51) Int. Cl. A61M 37/00

(52) U.S. Cl. 604/82

(57) ABSTRACT

A mixing device comprising a body portion, a removable tip portion, wherein said tip portion comprises an opening and a filter portion, wherein said filter portion comprises four filters. The mixing device may also comprise a plunger portion adapted for insertion into said body portion, wherein said plunger portion comprises a first plunger, wherein said first plunger comprises a mixing element, wherein said mixing element comprises a series of blades and said blades being disposed of within said body portion, and a second plunger, wherein said second plunger comprises a compression disk, and wherein a portion of a first plunger is disposed of within a second plunger, and wherein said first plunger operates independent of said second plunger.

### CROSS-REFERENCE TO RELATED APPLICATIONS

This application is a continuation in part of U.S. patent application Ser. No. 09/877,171 filed on Jun. 11, 2001 claiming priority to U.S. Provisional Patent Application No. 60/241,434 filed on Aug. 7, 2001, the disclosure of which is herein incorporated by reference in its entirety.

### BACKGROUND OF THE INVENTION

1. Field of the Invention

This invention relates to the field of mixing devices, specifically those devices used in the preparation of synthetic bone being comprised of a synthetic or natural bone powder combined with a liquid.

2. Description of the Related Art

There are a variety of synthetic bone powders that may consist of hydroxyapatite, calcium phosphate, calcium carbonate, or other similar biointegratable compounds. These powders may be mixed together with a liquid, such as sodium phosphate, water, or saline. After mixed together, the composition hardens to form a material similar to natural human bone that may be able to adhere to damaged bone sites, prosthetic devices, or other similar uses.

A shortcoming associated with this process is that the composition begins to harden and solidify, or crystallize, once the liquid is introduced to the powder, and therefore the mixing of the synthetic bone and the liquid must be accomplished within a specific time frame and with fastidious precision. Another shortcoming of the related art is that the composition may consist of sporadic dry spots within the composition that may not cure with the necessary bonding strength, and such that its application to the damaged bone site may not fully integrate with the damaged bone or whereby the composition may break apart. Still another shortcoming may be that too much solution may be added to the powder, and therefore the mixture may become wet such that after crystallization the composition may have weak bonds between powder particles due to air pockets within the composition from the evaporation of the excess liquid. Furthermore, the over wetting of the powder may prevent the mixture from drying and adhering to the site in a minimal amount of time. Because the composition begins to crystallize and cure immediately, and since the procedure may occur while the patient is being operated on, the mixing must be done accurately and quickly the first time such that the composition may be applied with minimal trauma to the patient, and whereby the composition cures with sufficient bonding strength to maintain its structure.

Traditional methods of mixing provide for a predetermined amount of synthetic bone to be mixed with a predetermined amount of liquid so that the resulting composition exhibits the required properties for curing and strength that results from a certain solution to powder ratio. These methods may be less favorable in the field because of the difficulty in ensuring that proper wetting is achieved, such that over wetting, under wetting, and/or leaving of dry spots may be controlled. Furthermore, due to this drawback, it may be difficult to obtain varying solution to powder ratios within the composition such that drying time can be controlled as to allow for increased or decreased drying time as specific instances may call for during its application.

### BRIEF SUMMARY OF THE INVENTION

Therefore a need has arisen for a mixing device and methods of mixing powders and liquids that overcomes these and other shortcomings of the related art. A technical advantage of the present invention may be to provide a mixing device that may allow for contained mixing of a powder and a liquid. Another technical advantage is to provide a mixing device that may expel excessive liquid from a composition, while said mixing device retains substantially all of the powder and an amount of liquid providing homogenous uniform dispersion of the powder within the liquid. Yet another technical advantage of the present invention is to provide a mixing device that may allow for the removal of at least a portion of the liquid from the mixing device. Moreover, the mixing device may allow for measured removal of a portion of liquid. Still yet another technical advantage of the present invention is that the mixing device may allow for measured dispensing of a mixed composition.

According to an embodiment of the present invention, a mixing device is described. The mixing device may comprise a body portion having a mixing cavity and a removable tip portion, in which tip portion may comprise an opening and a filter portion. In one embodiment, the filter portion may comprise four filters. In another embodiment, the mixing device may also comprise a plunger portion, whereby the plunger portion may be adapted for insertion into the body portion. Moreover, in this embodiment, the plunger portion may comprise a first plunger, in which the first plunger may comprise a mixing element. In one embodiment, the mixing element may comprise a bladed disk, wherein said disk may be disposed of within said body portion. The plunger portion may also comprise a second plunger, in which the second plunger may comprise a compression disk. According to this embodiment, a portion of the first plunger may be disposed of within the second plunger, and such that the first plunger may operate independent of the second plunger.

According to another embodiment of the present invention, a method of mixing a powder and a liquid that may result in a desired powder to liquid ratio is described. The method may comprise the step of adding a known amount of a powder to a mixing chamber and a volume of a liquid to the mixing chamber, in which the volume of the liquid may exceed the required volume of the liquid than that may be necessary to form the composition exhibiting an intended ratio of powder to liquid. The method also may comprise the step of mixing together the powder and the liquid within the mixing chamber, such that the mixing may form excessively wet composition, thus ensuring homogeneous uniform dispersion of the powder within the liquid. The method may further comprise the step of relieving a volume of a liquid from the supersaturated composition within the mixing chamber, such that a mixed composition retained in the mixing chamber may result that exhibits a desired ratio of powder to liquid.

Other objects, features, and advantages will be apparent to persons of ordinary skill in the art in view of the following detailed description of the invention and the accompanying drawings.

### BRIEF DESCRIPTION OF THE SEVERAL VIEWS

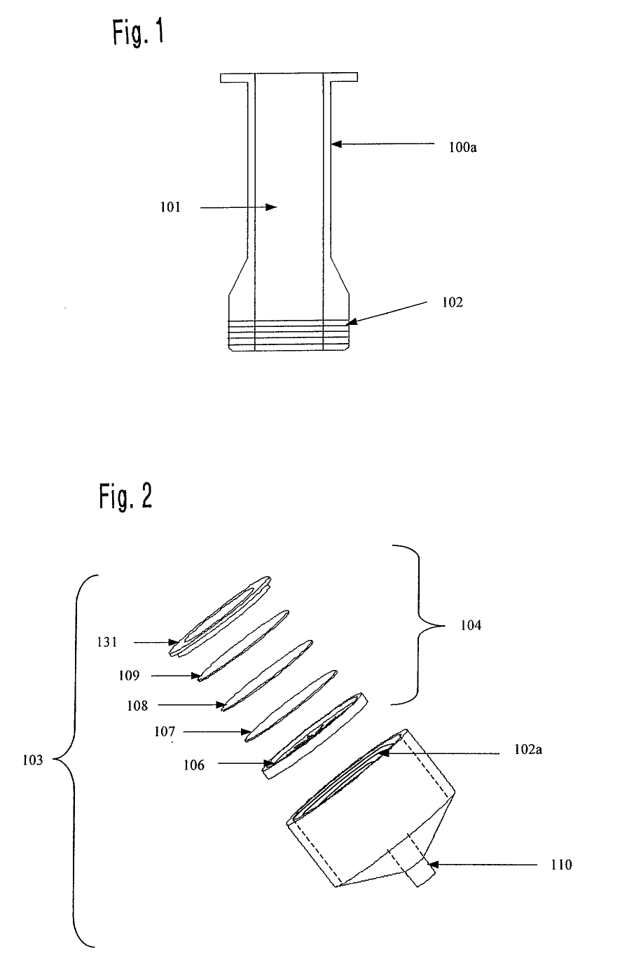

FIG. 1 depicts a body portion of the present invention according to a preferred embodiment.

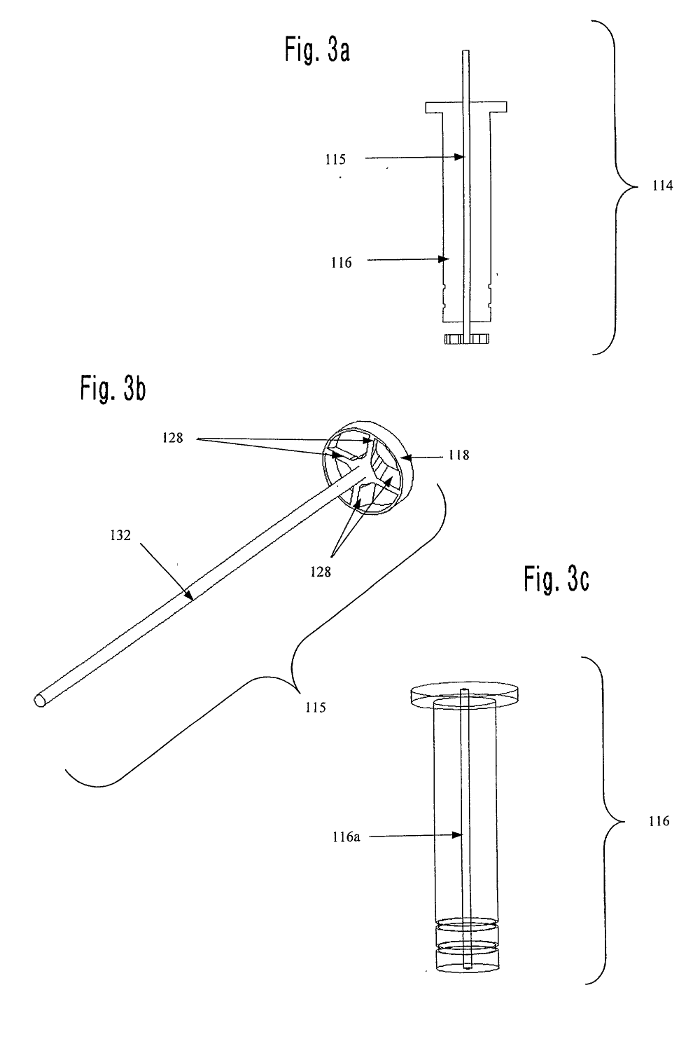

FIG. 2 depicts an exploded view of a filter system of the present invention according to a preferred embodiment.

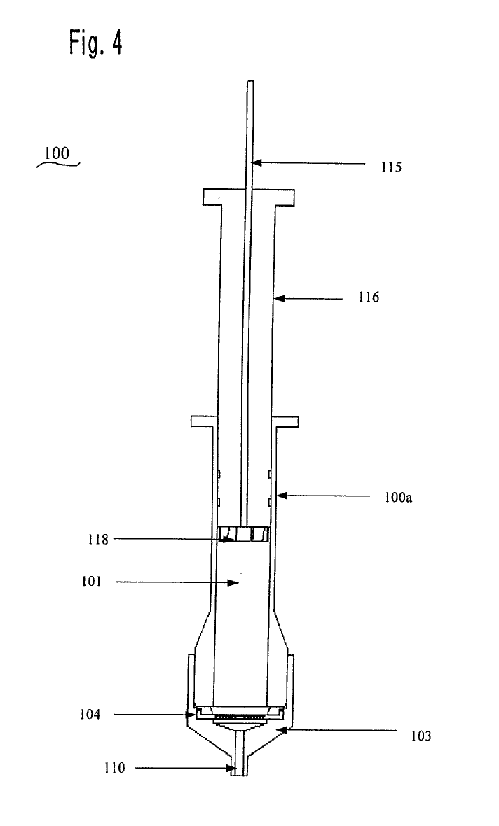

FIG. 3a depicts the plunger portion according to a preferred embodiment of the present invention.

FIG. 3b depicts a first plunger according to a preferred embodiment of the present invention.

FIG. 3c depicts a second plunger according to a preferred embodiment of the present invention.

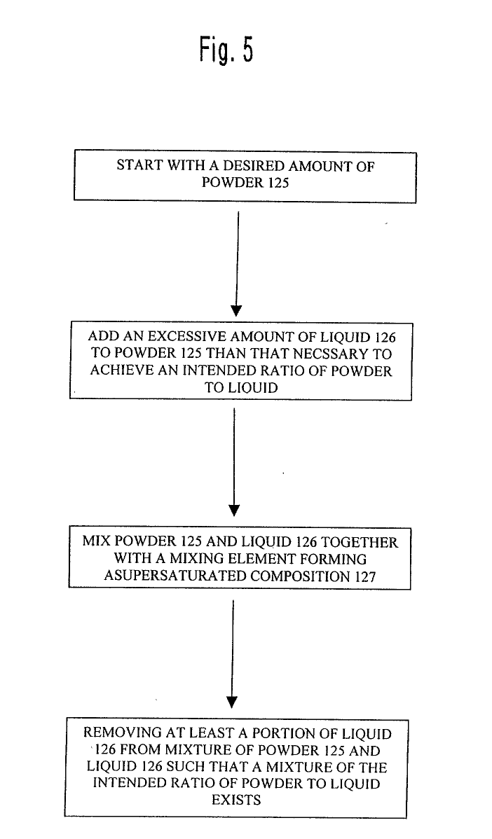

FIG. 4 depicts a mixing device according to a preferred embodiment of the present invention.

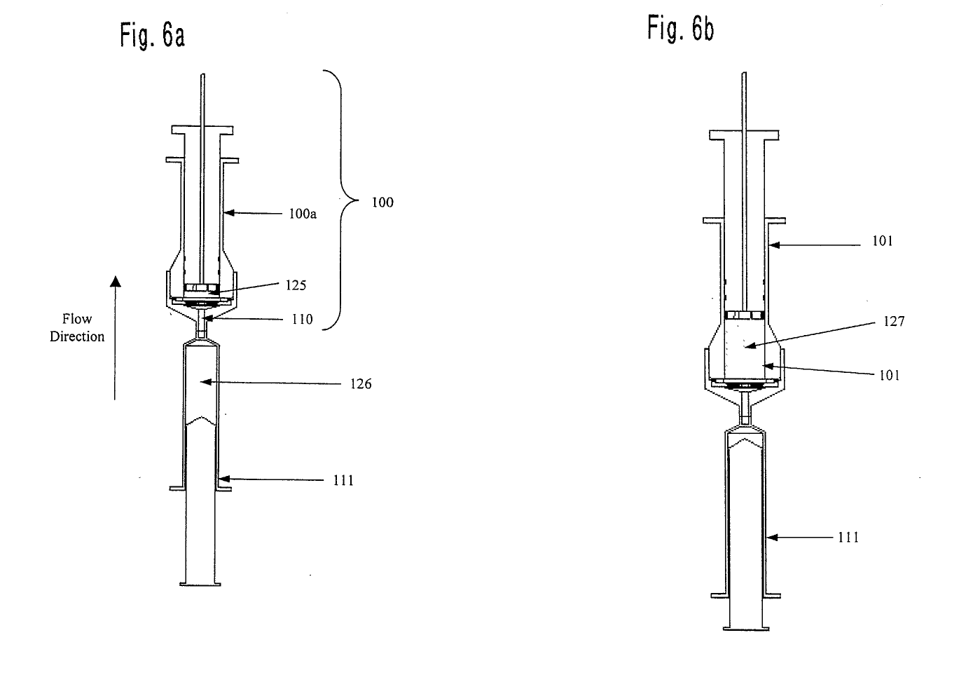

FIG. 5 depicts the steps comprising a method of mixing a powder and liquid together according to a preferred method of the present invention.

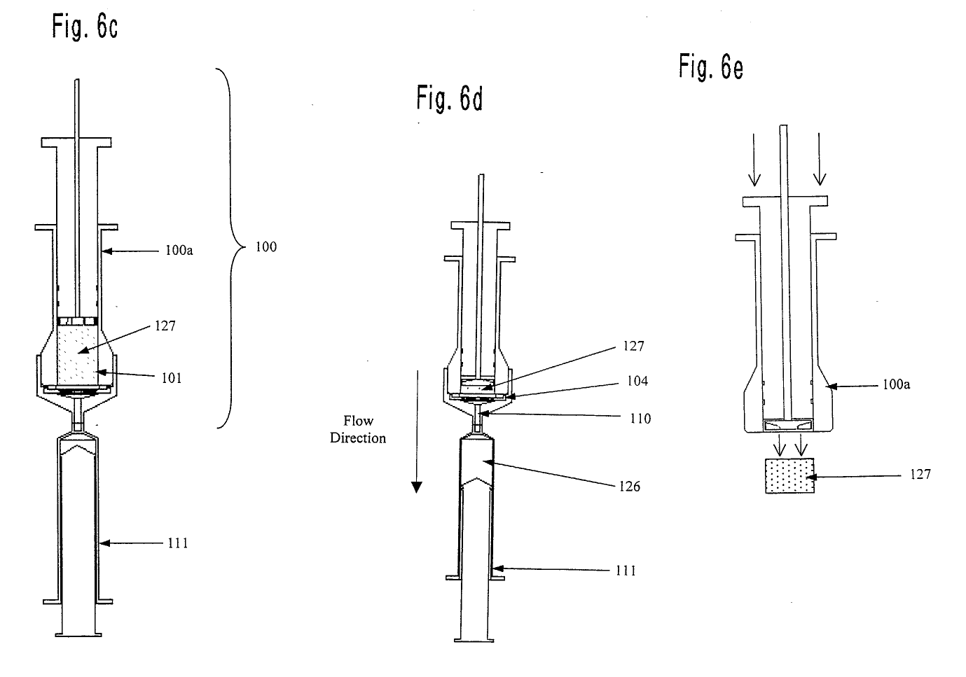

FIG. 6a depicts the mixing device according to the preferred method of use pursuant to the present invention.

FIG. 6b depicts the mixing device according to the preferred method of use pursuant to the present invention.

FIG. 6c depicts the mixing device according to the preferred method of use pursuant to the present invention.

FIG. 6d depicts the mixing device according to the preferred method of use pursuant to the present invention.

FIG. 6e depicts the mixing device according to the preferred method of use pursuant to the present invention.

### DETAILED DESCRIPTION OF THE INVENTION

Preferred embodiments of the present invention and their advantages may be understood by referring to FIGS. 1-6e, like numerals being used for like corresponding parts in the various drawings.

Referring to FIGS. 1, 2, and 4, a mixing device 100 for use in the mixing of a composition is described. Mixing device 100 may comprise a body portion 100a, which may comprise a hollow cavity 101 formed therein, such that hollow cavity may be adapted to hold a composition of at least a liquid and a powder. For example, hollow cavity 101 may be adapted for holding a synthetic bone powder and a liquid. For example, said synthetic powder may be BoneSource® and the liquid may be sodium phosphate solution. According to an embodiment, body portion 100a may be clear, whereby any contents within body portion 100a may be viewed from the outside of body portion 100a. In an embodiment, body portion 100a may be comprised of threading 102 covering at least a portion of body portion 100a.

In any of the above-described embodiments of the present invention, mixing device 100 may also comprise a tip portion 103, which may cover a portion of body portion 100a. For example, tip portion 103 may be a cap, such that tip portion 103 may be attachable to body portion 100a, or alternatively, tip portion 103 may be removable. In an embodiment, tip portion 103 may comprise threading 102a. According to this embodiment, threading 102a may be adapted to attach to threading 102 of body portion 100a.

In any of the above-described embodiments of the present invention, tip portion 103 may also comprise opening 110 formed therethrough. Opening 110 may permit the inflow of a substance to cavity 101, or alternatively, the outflow of a substance from cavity 101. In this embodiment, opening 110 may be adapted for connection to a fluid supply device, whereby a volume of fluid may be added to cavity 101 via said fluid supply device. In this embodiment, opening 110 may be adapted for connectivity to any know device for transmitting fluid from one source to a second source. For example, said fluid supply device may be a syringe, an irrigation tube, or a fluid pump. In an embodiment, not shown, opening 110 may further be comprised of a first threading, whereby said first threading may be adapted for the connectivity of said fluid supply device having a second threading adapted to mate with said first threading.

In any of the above-described embodiments of the present invention, tip portion 103 may further comprise a filter portion 104, wherein filter portion 104 may comprise at least one filter. For example, filter portion 104 may be comprised of a first filter 106, a second filter 107, a third filter 108, a fourth filter 109, and filter ring 131. Filter ring 131 may serve as a method of retaining filter 106, filter 107, filter 108 and filter 109 in a fixed filter portion 104. According to this embodiment, first filter 106 may have a porosity of about 0.1 mm-0.5 mm, second filter 107 may have a porosity of about 10-200 μm, third filter 108 may have a porosity of about 0.2 μm, and fourth filter 109 may have a porosity of about 10-200 μm. In another embodiment, filter portion 104 may be removable from tip portion 103, and whereby the removal of filter portion 104 may facilitate the inflow, or alternatively, the outflow of a substance through opening 110.

In any of the above-described embodiments of the present invention, mixing device 100 may also comprise plunger portion 114, as depicted in FIGS. 3a-3c, and FIG. 4. According to an embodiment of the present invention, plunger portion 114 may be adapted for insertion into body portion 100a, and wherein plunger portion 114 may comprises multiple plungers. For example, plunger portion 114 may be comprised of a first plunger 115. Plunger 115 may be comprised of shaft 132 and a mixing element 118. In one embodiment, mixing element 118 may be comprised of at least one mixing blade 128. For example, mixing element may comprise four mixing blades 128. Furthermore, mixing blades 128 may be set to an angle θ relative to the vertical plane of shaft 132, and such that angle θ may facilitate both the churning and the movement of a composition within cavity 101. For example, mixing blades 128 may be set an angle θ of about 20°. In another embodiments, not shown, mixing element 118 may comprise a perforated disk having a series of openings formed therethrough, or mixing element 118 may be comprised of a spoked disk. In any of the above-described embodiments, at least a portion of mixing element 118 may be disposed of within cavity 101.

In any of the above-described embodiments of the present invention, plunger portion 114 may further comprise a second plunger 116. Plunger 116 may be comprised of channel 116a comprising a pathway extending the length of plunger 116. According to this embodiment, a portion of second plunger 116 may be disposed of around at least a portion of first plunger 115. For example, plunger 115 may be disposed of within channel 116a. According to this embodiment, first plunger 115 may operate independent of second plunger 116, such that the movement of plunger 115 may not cause the movement of plunger 116. In still another embodiments, not shown, second plunger 116 may further comprise at least one locking groove, such that said locking groove may hold second plunger 116 at fixed points within said body portion 100a. For example, in operation, upon second plunger 116 being inserted into cavity 101, said locking groove may engage a locking bar, such that second plunger 116 is retained at a fixed position that requires an additional force to disengage said locking bar, wherein freedom of movement for second plunger 116 may be restored. In another embodiment, also not shown, second plunger 116 may comprise a first threaded portion, and wherein body portion 100a may comprises a reciprocal second threaded portion, and wherein said first threaded portion and said second threaded portion may engage as a torque drive that may allow greater application force within body cavity 101, then that which may result from the use of direct downward force only.

In any of the above-described embodiments of the present invention, body portion 100a may also be adapted for use with an actuation portion (not shown). According to this embodiment, said actuation portion may comprise a series of shaped connectors that may be adapted for receiving body portion 100a, and such that body portion 100a may be retained in a fixed position upon being connected with said actuation portion. Said actuation portion may further comprise a hammer portion, and wherein said hammer portion may be adapted to contact at least a portion of plunger portion 114. Further according to this embodiment, said actuation portion may also comprise a trigger, wherein said trigger may be adapted for controlling the activation of said hammer portion, and wherein said hammer portion may then apply force to plunger portion 114, whereby the insertion of plunger portion 114 into cavity 101 may be facilitated.

FIG. 4 depicts mixing device 100 according to its exemplary embodiments. Mixing device 100 may comprise body portion 100a. Mixing device 100 may also comprise cavity 101. Mixing device 100 may further comprise tip portion 103, wherein tip portion 103 may be comprised of filter system 104 and opening 110. Moreover, mixing device 100 comprises a plunger 116 and plunger 115, wherein plunger 115 may further be comprised of mixing element 118, and whereby at least a portion of plunger 115 may be disposed of within plunger 116.

FIG. 5 and FIGS. 6a-6e describe a method of mixing a powder 125 and a liquid 126 together to form a composition 127, having an intended and predetermined ratio of powder 125 to liquid 126, according to a preferred method of this invention. According to a preferred embodiment of this method, composition 127 may be formed from a mixture of powder 125 and liquid 126, wherein the volume of liquid 126 may exceed the necessary amount that may be required to achieve the intended ratio of powder to liquid, and such that homogeneous uniform dispersion may be achieved. For example, the method of mixing may result in a composition having a 1-to-1 ratio of powder to liquid, whereby the beginning ratio of powder to liquid may be 1-to-4. The method according to the preferred embodiments may consist of the steps of first mixing together a known amount of powder 125 to an amount of liquid 126, wherein the volume of liquid 126 may exceed the required amount necessary to form composition 127, such that composition 127 may result from an excessive amount liquid 126 being combined with powder 125. In an embodiment of the present method, liquid 126 may be introduced to powder 125 through any known device that may carry or deliver a liquid from a first device to a second device. For example, liquid 126 may be introduced to powder 125 through the use of a syringe 129, or liquid 126 may be introduced to powder 125 through an irrigation tube. Still in another embodiment, liquid 126 may be introduced at the same time as the introduction of powder 125.

In any of the above-described embodiments of the present invention, the preferred method of mixing may also comprises the additional step of mixing powder 125 and liquid 126 together, whereby the resulting composition 127 may be a homogeneous uniform dispersion of powder 125 within liquid 126. For example, powder 125 and liquid 126 may be whipped together, and wherein powder 125 may be substantially suspended within liquid 126 and be substantially saturated. According to an exemplary embodiment, the step of mixing powder 125 and liquid 126 together may be facilitated by the use of a mixing element 118, wherein mixing element 118 may be comprised of any known mixing apparatus. For example, mixing element 118 may be comprised of a wheel, a perforated disk, or at least one mixing blade 128. According to this embodiment, powder 125 and liquid 126 may be mixed together utilizing a mixing element 118, wherein the mixing element 118 may pushed and pulled within powder 125 and liquid 126 mixture, and wherein mixing element 118 may additionally be spun within powder 125 and liquid 126, whereby said spinning may be accomplished by manually spinning mixing element 118. Alternatively, said spinning may be accomplished through mechanically spinning mixing element 118, whereby mixing element 118 may be adapted for connection to a drill or other electronic spinning device, such that high rotational energy may be applied to powder 125 and liquid 126, and such that whipping of powder 125 and liquid 126 may result from the high rotational energy. Furthermore, high rotational energy may transmit increased heat into the mixture of powder 125 and liquid 126, whereby the introduction of heat to the mixture cause decreased curing time for composition 127.

In any of the above-described embodiments of the present invention, the mixture of powder 125 and liquid 126 may be relieved of at least a portion of excess liquid 126, such that a composition 127 results having the properties of simple hydration of powder 125 by liquid 126. For example, excessive liquid 126 may be relieved from cavity 101 through forcing the mixture against a filtering mechanism, whereby said filtering mechanism permits the passage of excessive liquid 126, however, said filtering mechanism retains powder 125 in a hydrated state, and whereby said composition 127 may exhibit characteristics of an intended powder to liquid ratio. Through the removal of excess liquid 126 from composition 127 may increase the bonding strength of powder 125 during the crystallization of composition 127 due to the decrease in the amount of liquid 126 that may be required to evaporate from composition 127 and wherefrom air pockets may be significantly reduced.

In any of the above-described embodiments of the present invention, this method of mixing may further comprise the step of removing at least a portion of liquid 126, in a series of at least two mixing sequences, according to the aforementioned method of mixing. For example, super hydrated composition 127 may be relieved of a portion of said liquid and supersaturated composition 127 may then be remixed, prior to a subsequent relieving of a further excess of liquid 126.

According to other embodiments of the present invention, the method of mixing a composition may further comprise the step of removing composition 127 from a mixing device. According to this embodiment, the method may comprise the step of removing composition 127 in a precise method, such as through the pressing of composition through a small opening.

In operation, and according to the above-described method, FIGS. 6a-6e depict mixing device 100 according to any of the above-described embodiments may be used to perform the described method of mixing powder 125 and liquid 126 to form composition 127. For example, powder 125 may be placed within mixing device 100. The method may next comprise the step of adding liquid 126 to cavity 101. Liquid 126 may be added to cavity 101 through opening 110, and whereby liquid 126 may be transferred through a fluid supply 111 adapted to be connected to opening 110, such as a syringe or an irrigation tube. According to the above-mentioned method of the present invention, the volume of liquid 126 added to powder 125 may exceed the volume that may be required to form composition 127 that may ensure that the composition 127 achieves homogeneous uniform dispersion after powder 125 and liquid 126 are mixed together, and such that composition 127 may be characterized as being in a super hydrated state.

In any of the above-described embodiments of method of the present invention, composition 127 may then be mixed such as to achieve homogeneous uniform dispersion. For example, plunger 115 may be spun, whereby mixing element 118 is rotated within cavity 101, and such that powder 125 and liquid 127 may be substantially mixed together forming composition 127. In this embodiment, mixing element 118 may be spun through manually rotating plunger 115. Furthermore, plunger 115 may be spun at an increased velocity. For example, plunger 115 may be adapted for connection to a drill (not shown), whereby said drill may provide for the increased revolutions of mixing element 118. Still yet in another embodiment, plunger 115 may be repeatedly inserted and withdrawn from cavity 101, such that mixing and agitation of powder 125 and liquid 126 may be further facilitated.

In any of the above-described embodiments of the method of the present invention, cavity 101 may then be relieved of any excess liquid 126 comprising composition 127 in a super hydrated state. In a preferred embodiment, cavity 101 may be relieved of excess liquid 126 through the application of plunger 116 to an inserted position, and such that pressure may be applied to composition 127 within cavity 101 forcing composition 127 against filter portion 104 and out of cavity 101 through opening 110. According to this embodiment, filter portion 104 may permit the passage of a portion fluid 126, but may retain substantially all of powder 125, and whereby the remaining liquid 126 and powder 125 may be in a ratio such that powder 125 may be completely hydrated with only the required volume of liquid 126 necessary to ensure complete hydration. For example, the ratio of powder to liquid may be 1-to-4 in a super hydrated state and in a homogeneous uniform dispersion, then after mixing and the removal of excess liquid, then a 1-to-1 ratio of powder to liquid may be retained within cavity 101. According to another embodiment of the present invention, the method of removing excess liquid 126 from cavity 101 may be accomplished in a series of successive combinations of mixing composition 127 and removing at least a portion of liquid 126.

In any of the above-described embodiments of the method of the present invention, an actuation portion (not shown) adapted for connection to mixing device 100 may be used to facilitate the removal of fluid 126 through mechanically applying pressure against composition 127. For example, actuation portion may be adapted for holding body portion 100a, and whereby the depressing of a trigger activates the forward movement of plunger portion 114 causing pressure within cavity 101.

In any of the above-described embodiments of the method of the present invention, composition 127 may then be removed from cavity 101. For example, tip portion 103 may be removed from body portion 100a, and whereby removal of composition 127 may be accomplished from body cavity 101 from where tip portion 103 had been removed and plunger 116 is compressed. Alternatively, tip portion 103 may be removed and filter portion 104 removed from tip portion, and then replacing tip portion 103 on body portion 100a, whereby composition 127 may be removed by forcing composition 127 through opening 110.

In any of the above-described embodiments, the resulting composition 127 may comprise a mixture of powder 125 and liquid 126, wherein the ratio of powder to liquid may be that of a predetermined and intended ratio, such as to exhibit the properties for eliciting an intended crystallization and curing time to achieve the intended strength of the bonds for composition 127.

While the invention has been described in connection with preferred embodiments, it will be understood by those of ordinary skill in the art that other variations and modifications of the preferred embodiments described above may be made without departing from the scope of the invention. Other embodiments will be apparent to those of ordinary skill in art from a consideration of the specification or practice of the invention disclosed herein.

What is claimed is:

1. A mixing device comprising:
a body portion, wherein said body portion comprises a hollow cavity,
a tip portion, wherein said tip portion comprises a filter portion; and
a plunger portion adapted for insertion into said body portion, wherein said plunger portion comprises multiple plungers, wherein a portion of a first plunger is disposed of within a second plunger, and wherein said first plunger operates independent of said second plunger.

2. The device of claim 1, wherein said body portion is adapted for use with an actuation portion, wherein said actuation portion comprises shaped connectors that are adapted for receiving said body portion, a trigger portion and a hammer portion, and wherein said trigger portion is adapted for controlling the activation of said hammer portion, and wherein said hammer is adapted for sliding said plunger portion.

3. The device of claim 1, wherein said body portion is clear.

4. The device of claim 1, wherein said tip portion comprises an opening.

5. The device of claim 4, wherein said opening is adapted for connectivity to a fluid supply device.

6. The device of claim 5, wherein said opening further comprises a first threaded portion adapted for the connectivity of said fluid supply device having a second threaded portion adapted to mate with said first threaded portion.

7. The device of claim 5, wherein said fluid supply device comprises a syringe.

8. The device of claim 5, wherein said fluid supply device comprises an irrigation tube adapted for connectivity to said opening.

9. The device of claim 4, wherein said opening is adapted for expelling a substance.

10. The device of claim 9, wherein said opening is adapted for attachment to a fluid receiving device.

11. The device of claim 10, wherein said fluid receiving device is a syringe.

12. The device of claim 10, wherein said fluid receiving device is an irrigation tube.

13. The device of claim 1, wherein said tip portion is removable from said body portion, and wherein said filter portion is removable from said tip portion.

14. The device of claim 1, wherein said filter portion comprises at least one filter.

15. The device of claim 14, wherein the number of filters that said filter portion comprises is four.

16. The device of claim 15, wherein said filter portion comprises a first filter, wherein said first filter comprises a porosity of about 0.1 mm-0.5 mm.

17. The device of claim 15, wherein said filter portion comprises a second filter, wherein said second filter comprises a porosity of about 10-200 μm.

18. The device of claim 15, wherein said filter portion comprises a third filter, wherein said third filter comprises a porosity of about 0.2 μm.

19. The device of claim 15, wherein said filter portion comprises a fourth filter, wherein said fourth filter comprises a porosity of about 10-200 μm.

20. The device of claim 1, wherein said first plunger comprises a mixing element, wherein said mixing element is disposed of within said body portion

21. The device of claim 20, wherein said mixing element comprises a series of blades.

22. The device of claim 21, wherein said blades are set at an angle.

23. The device of claim 20, wherein said mixing element comprises a perforated disk.

24. The device of claim 20, wherein said mixing element comprises a disk having at least one spoke.

25. A mixing device comprising:
a body portion comprising a body portion;
a removable tip portion, wherein said tip portion comprises an opening and a removable filter portion, wherein said filter portion comprises four filters; and
a plunger portion adapted for insertion into said body portion, wherein said plunger portion comprises a first plunger, wherein said first plunger comprises a mixing element, wherein said mixing element comprises a series of blades and said blades being disposed of within said body portion, and a second plunger, wherein said second plunger comprises a compression disk, and wherein a portion of a first plunger is disposed of within a second plunger, and wherein said first plunger operates independent of said second plunger.

26. The device of claim 25, wherein said body portion is clear.

27. The device of claim 25 further comprising an actuation portion, wherein said actuation portion comprises shaped connectors adapted for receiving said body portion, a trigger portion and a hammer portion, and wherein said trigger portion is adapted for controlling the activation of said hammer portion, and wherein said hammer is adapted for sliding said plunger portion.

28. The device of claim 25, wherein said filter portion comprises a first filter, wherein said first filter comprises a porosity of about 0.1 mm-0.5 mm.

29. The device of claim 25, wherein said filter portion comprises a second filter, wherein said second filter comprises a porosity of about 10-200 μm.

30. The device of claim 25, wherein said filter portion comprises a third filter, wherein said third filter comprises a porosity of about 0.2 μm.

31. The device of claim 25, wherein said filter portion comprises a fourth filter, wherein said fourth filter comprises a porosity of about 10-200 μm.

32. The device of claim 25, wherein said first plunger comprises a first threaded portion, and wherein said body portion comprises a reciprocal second threaded portion, and wherein said first threaded portion and said second threaded portion are adapted to cause added pressure within said body portion through the rotation of said first plunger within said body portion.

33. A method of mixing a powder and a liquid to form a composition having a desired ratio of said powder to said liquid, wherein said method comprises the steps of:
adding a known amount of said powder to a mixing chamber and an amount of said liquid to said cavity, wherein said liquid exceeds the required amount to form said composition exhibiting said desired ratio,
wherein said liquid and said powder are then mixed together within said mixing chamber forming a super hydrated composition, and
wherein said composition within said mixing chamber is then relieved of excess liquid, wherein a mixed composition results exhibiting said desired ratio of powder to liquid is retained within said mixing chamber.

34. The method of claim 33, wherein said composition is comprised of said powder in homogeneous uniform dispersion within said liquid.

35. The method of claim 33, wherein said composition is super hydrated through the addition of an excess amount of said liquid.

36. The method of claim 33 further comprising the step of removing at least a portion of said liquid, wherein said super hydrated composition is relieved of excessive liquid in a series of at least two mixing sequences, wherein said supersaturated composition is relieved of a portion of said liquid, and wherein said supersaturated composition is remixed prior to a subsequent relieving of a further excess of said liquid.

37. The method of claim 33 further comprising of the step of removing said composition from said mixing chamber.

38. The method of claim 37, wherein said mixing is accomplished utilizing the device of claim 25.

39. The method of claim 38, wherein said liquid is added to said mixing chamber via a syringe.

40. The method of claim 38, wherein said liquid is added to said mixing chamber via an irrigation tube.

41. The method of claim 38 comprising the step of removing said composition from said mixing chamber leaving an exposed tip portion, wherein said tip portion is removed and said composition is removed through said exposed tip portion.

42. The method of claim 39 further comprising the step of attaching a second tip portion, wherein said second tip portion comprises a second opening with no filter portion disposed of within said tip portion, and expelling said composition through said second opening.

43. The method of claim 33, wherein said powder is a synthetic bone powder.

44. The method of claim 33, wherein said powder is a natural bone powder.

45. The method of claim 33, wherein said liquid is sodium phosphate solution.

46. The method of claim 33, wherein said liquid is water.

47. The method of claim 33, wherein said liquid is saline solution.
